(19) United States
(12) Patent Application Publication
(10) Pub. No.: US 2020/0300844 A1
(43) Pub. Date: Sep. 24, 2020

(54) METHODS, SYSTEMS AND DEVICES FOR DETECTING INFLAMMATION

(71) Applicant: Stellenbosch University, Stellenbosch, Western Cape Province (ZA)

(72) Inventors: Etheresia Pretorius, Stellenbosch (ZA); Anna Martha Engelbrecht, Zevendal, Kuilsriver (ZA); Willem Jacobus Perold, Somerset West (ZA); Deon Pieter Neveling, Hilary, Durban (ZA)

(21) Appl. No.: 16/763,452

(22) PCT Filed: Nov. 13, 2018
(86) PCT No.: PCT/IB2018/058904

§ 371 Date: May 12, 2020

(30) Foreign Application Priority Data
Nov. 13, 2017 (GB) 1718708.9

(51) Int. Cl. G01N 33/543; G01N 33/551

(52) U.S. Cl. CPC G01N 33/5438; G01N 2800/7095; G01N 2610/00; G01N 33/551

(57) ABSTRACT

A method, system, test strip, point-of-care device and computer-implemented method for detecting a level of inflammation in a subject is provided. The level of inflammation is detected by contacting a biological sample obtained from the subject with a serum amyloid A (SAA) capture agent. The capture agent is secured to a substrate and is configured to emit a signal upon binding to SAA. The signal is detected and a result indicating the level of inflammation in the subject is output.

### FIELD OF THE INVENTION

This invention relates to methods, systems and devices for the detection of inflammation in a subject. In particular, it relates to methods, systems and devices for the detection and quantification of the biomarker, serum amyloid A, which is associated with inflammation.

### BACKGROUND TO THE INVENTION

The global disease burden is continuing to shift away from communicable diseases to non-communicable diseases such as diabetes, atherosclerosis, Alzheimer's disease, cardiovascular disease and cancer—all of which are linked to chronic low-grade inflammation. Furthermore, about 80% of people dying from these diseases now live in the developing world, which holds a particular danger for health systems of developing countries which are already under-resourced and over-stretched. It is thus essential to investigate possible markers which link inflammation to these diseases and to develop low cost methods of early detection.

Several pro-inflammatory gene products have been identified as mediators of disease, one example being serum amyloid A (SAA). SAA is a generic term for a family of acute phase proteins synthesised by the liver which are mainly regulated by inflammation associated cytokine-peptide hormone signals. Inflammation resulting from cancer, cardiovascular disease, rheumatoid arthritis, bacterial infection, and tissue damage, may cause SAA levels to rise 1000-fold, and these elevated levels may be diagnostic of an inflammatory disease.

Currently, SAA levels can be detected using enzyme-linked immunosorbent assays (ELISA) and mass spectrometry (MS). However, these methods are poorly sensitive, extremely expensive and time-consuming. This may limit their application, particularly in under resourced clinical contexts.

There is therefore a need for a means of detecting inflammation in a subject that addresses the aforementioned problems, at least to some extent.

The preceding discussion of the background to the invention is intended only to facilitate an understanding of the present invention. It should be appreciated that the discussion is not an acknowledgment or admission that any of the material referred to was part of the common general knowledge in the art as at the priority date of the application.

### SUMMARY OF THE INVENTION

According to a first aspect of the invention, there is provided a method for detecting a level of inflammation in a subject, the method comprising: contacting a biological sample obtained from the subject with a serum amyloid A (SAA) capture agent, which is secured to an electrically conductive polymeric nanofibre and which is configured to emit an impedance signal upon binding to SAA; detecting a signal; and outputting a result indicating a level of inflammation in the subject based on the detected signal, characterised in that the nanofibre contains metal nanoparticles.

The method may include comparing the signal with a predetermined reference value to diagnose the level of inflammation in the subject. The level of inflammation may be indicative of a disease selected from the group consisting of: cancer, atherosclerosis or increased vascular risk, rheumatoid arthritis, Alzheimer's disease, amyloidosis, giant cell arthritis, coronary heart disease, Behçet's disease, sickle cell anemia, immune thrombocytopaenic purpura, HIV, stroke, pre-eclampsia, inflammation-associated thrombosis, type II diabetes, and infection. The level of inflammation may also be indicative of a degree of disease progression in the subject.

The capture agent may be selected from the group consisting of: thioflavins, NIAD-4 (2-[[5′-(4-hydroxyphenyl)[2,2′-bithiophen]-5-yl]-methylene]-propanedinitrile), luminescent conjugated oligothiophene (LCO) markers, SAA-binding antibodies or antibody fragments, high density lipoprotein (HDL), affibodies, ankyrin repeat proteins, armadillo repeat proteins, nucleic acid aptamers, modified nucleic acid aptamers, peptides, modified peptides, carbohydrate ligands, and synthetic ligands.

The capture agent may be secured to the metal nanoparticles by a linker. The linker may include a mercapto functionality at a first end thereof and an alkanoic acid at an opposite second end thereof. The linker may be a self-assembled monolayer (SAM), which may be 3-mercaptopropanoic acid or poly(ethylene glycol) 2-mercaptoethyl ether acetic acid. The metal nanoparticles may be gold nanoparticles.

The nanofibre may comprise a non-electrically conductive first polymer and an electrically conductive second polymer. The nanofibre may be formed by electrospinning the first and second polymers together with the metal nanoparticles.

The nanofibre may be included in a test strip which may be configured for use with a point-of-care device, such as a hand held device, and the test strip may be a single-use disposable test strip or a multiple-use test strip. Alternatively, the nanofibre may be integrally formed with a sample receiving surface of a point-of-care device, such as a hand held device, and may be capable of being successively used with multiple samples.

The method may further include amplifying the detected signal to produce an amplified signal; converting the amplified signal to a digital signal; recording, analysing and/or processing the digital signal; determining an amount of SAA in the sample; and assigning a level of inflammation based on the amount of SAA detected.

The biological sample may be whole blood, blood plasma, blood serum, urine, saliva, sputum, or tissue obtained from a biopsy.

According to a second aspect of the invention, there is provided a system for detecting a level of inflammation in a subject according to the method defined above, the system including: an electrically conductive polymeric nanofibre for receiving a biological sample from the subject thereon; a capture agent secured to the nanofibre for binding SAA in the sample, the capture agent being configured to emit an impedance signal upon binding to SAA; a sensor in communication with the nanofibre for detecting the emitted signal; and an output member in communication with the sensor configured to output a result indicating a level of inflammation in the subject based on the detected signal, characterised in that the nanofibre contains metal nanoparticles.

The capture agent, nanofibre and biological sample may be as defined above.

According to a third aspect of the invention, there is provided a test strip for use in detecting a level of inflammation in a subject, the test strip including: an electrically conductive polymeric nanofibre for receiving a biological sample from the subject thereon, and a capture agent secured to the nanofibre for binding SAA in the sample, the capture agent being configured to emit an impedance signal upon binding to SAA when connected to an electrical circuit, the signal being indicative of the level of inflammation in the subject, characterised in that the nanofibre contains metal nanoparticles.

The capture agent, nanofibre and biological sample may be as defined above.

The test strip may be configured for use with a point-of-care device, such as a hand held device, and may be a single-use disposable test strip or a multiple-use test strip.

According to a fourth aspect of the invention, there is provided a point-of-care device for detecting a level of inflammation in a subject, the device including: a sample receiving zone for receiving and contacting a biological sample from the subject with an SAA capture agent, the capture agent being secured to an electrically conductive polymeric nanofibre containing metal nanoparticles and configured to emit an impedance signal upon binding to SAA when connected to an electrical circuit; a sensor configured to be operatively in communication with the nanofibre for detecting the signal; and an output member in communication with the sensor configured to output a result indicating a level of inflammation in the subject based on the detected signal.

The device may further include a processor for processing the signal. The processor may be configured to compare the signal with a predetermined reference value to diagnose the level of inflammation in the subject. The predetermined reference value may be one or more values on a standard curve.

The sensor may be selected from a a volt meter, an ammeter, an oscilloscope and a power meter.

According to a fifth aspect of the invention, there is provided a computer-implemented method for detecting inflammation in a subject, the method including: receiving an impedance signal from a sensor configured to detect binding of SAA in a biological sample to an SAA-binding capture agent, the capture agent being secured to an electrically conductive polymeric nanofibre and configured to emit an impedance signal upon binding to SAA, comparing the signal to a predetermined reference value to diagnose the level of inflammation in the subject, and outputting a result indicating the level of inflammation in the subject based on the signal, characterised in that the nanofibre contains metal nanoparticles.

The computer-implemented method may further include amplifying the signal to produce an amplified signal; converting the amplified signal to a digital signal; recording, analysing and/or processing the digital signal; determining an amount of SAA in the sample; and assigning a level of inflammation based on the amount of SAA detected.

According to a sixth aspect of the invention, there is provided a method for detecting a level of inflammation in a subject, the method comprising: contacting a biological sample obtained from the subject with a serum amyloid A (SAA) capture agent, which is secured to a substrate and which is configured to emit a signal upon binding to SAA, detecting the signal, and outputting a result indicating a level of inflammation in the subject based on the signal, characterised in that the substrate is a piezoelectric substrate and the signal is a piezoelectric signal.

The substrate may include a plurality of piezoelectric nanowires which may have ends thereof mounted on a semi conductive substrate and opposite free ends extending generally parallel in a direction substantially perpendicular to the semi conductive substrate, each nanowire may have the capture agent immobilised onto at least a portion of a surface of a free end thereof. Base portions of the nanowires may be coated with an insulating layer of material which may fill the spaces between the nanowires whilst the free ends remain substantially uncoated and uninsulated, and displacement of the nanowires owing to binding of SAA with the capture agent immobilised on the free ends may produce a piezoelectric signal.

At least a portion of the nanowires may be coated in gold and the capture agent may be secured to the gold via a linker. The linker may be provided by glutaraldehyde or by streptavidin.

The method may include comparing the signal with a predetermined reference value to diagnose the level of inflammation in the subject. The level of inflammation may be indicative of a disease selected from the group consisting of: cancer, atherosclerosis or increased vascular risk, rheumatoid arthritis, Alzheimer's disease, amyloidosis, giant cell arthritis, coronary heart disease, Behçet's disease, sickle cell anemia, immune thrombocytopaenic purpura, HIV, stroke, pre-eclampsia, inflammation-associated thrombosis, type II diabetes, and infection. The level of inflammation may also be indicative of a degree of disease progression in the subject.

The capture agent may be selected from the group consisting of: thioflavins, NIAD-4 (2-[[5′-(4-hydroxyphenyl)[2,2′-bithiophen]-5-yl]-methylene]-propanedinitrile), luminescent conjugated oligothiophene (LCO) markers, SAA-binding antibodies or antibody fragments, high density lipoprotein (HDL), affibodies, ankyrin repeat proteins, armadillo repeat proteins, nucleic acid aptamers, modified nucleic acid aptamers, peptides, modified peptides, carbohydrate ligands, and synthetic ligands.

The substrate may be included in a test strip which may be configured for use with a point-of-care device, such as a hand held device, and the test strip may be a single-use disposable test strip or a multiple-use test strip. Alternatively, the substrate may be integrally formed with a sample receiving surface of a point-of-care device, such as a hand held device, and may be capable of being successively used with multiple samples.

The method may further include amplifying the detected signal to produce an amplified signal; converting the amplified signal to a digital signal; recording, analysing and/or processing the digital signal; determining an amount of SAA in the sample; and assigning a level of inflammation based on the amount of SAA detected.

The biological sample may be whole blood, blood plasma, blood serum, urine, saliva, sputum, or tissue obtained from a biopsy.

According to a seventh aspect of the invention, there is provided a system for detecting a level of inflammation in a subject according to the method defined above, the system including: a substrate for receiving a biological sample from the subject thereon; a capture agent secured to the substrate for binding SAA in the sample, the capture agent being configured to emit a signal upon binding to SAA; a sensor in communication with the substrate for detecting the emitted signal; and an output member in communication with the sensor configured to output a result indicating a level of inflammation in the subject based on the detected signal, characterised in that the substrate is a piezoelectric substrate and the signal is a piezoelectric signal.

The capture agent, substrate and biological sample may be as defined above.

According to an eighth aspect of the invention, there is provided a test strip for use in detecting a level of inflammation in a subject, the test strip including a substrate for receiving a biological sample from the subject thereon, and a capture agent secured to the substrate for binding SAA in the sample, the capture agent being configured to emit a signal upon binding to SAA, wherein the signal is indicative of the level of inflammation in the subject, characterised in that the substrate is a piezoelectric substrate and the signal is a piezoelectric signal.

The capture agent, substrate and biological sample may be as defined above.

An embodiment of the invention will now be described, by way of example only, with reference to the accompanying drawings.

### BRIEF DESCRIPTION OF THE FIGURES

FIG. 1 is a schematic representation of the invention in which the capture agent is an antibody which is secured to a linker and which is configured to emit an impedance or piezoelectric signal upon binding to SAA.

FIG. 2 is a plan view of one embodiment of a test strip and device according to the invention for detecting SAA in a blood sample from a subject.

FIG. 3 is a schematic representation of an embodiment of a system according to the invention in which a test strip is analysed by a resistance detector in communication with a constant current generator, a processor, a memory component and an output member.

FIG. 4 is a circuit diagram illustrating the components of a circuit to which a substrate of a test strip is contactable. The circuit includes a nanofibre, a constant current generator, and a sensor in the form of a resistance detector. A processor is in communication with the constant current generator and resistance detector.

FIG. 5 is a section view of an embodiment of the system in which the substrate includes a plurality of piezoelectric nanowires.

FIG. 6 is a plan view of an embodiment of the test strip in which the nanofibre is in the form of a textile.

FIG. 7 is a plan view of an embodiment of the test strip in which the nanofibre is in the form of an elongate strand.

FIG. 8 is a perspective view of the test strip of FIG. 7 in proximity to a docking means of an inflammation measuring device. The docking means is configured to receive the test strip. An electrical circuit including a constant current generator and resistance detector is in communication with the docking means.

FIG. 9 is a perspective view of the test strip of FIG. 7 connected to an electrical circuit that includes a constant current generator and resistance detector.

FIG. 10 is a perspective view of the test strip of FIG. 6 approaching a docking means of an inflammation measuring device.

FIG. 11 is a flow diagram illustrating steps of a computer-implemented method according to the invention.

### DETAILED DESCRIPTION OF THE INVENTION

A method (100), system (200), test strip (300), device (400) and computer-implemented method (500) for detecting inflammation in a subject are herein described. These can be used to diagnose a level of inflammation in the subject. The level of inflammation can be indicative of the presence of a disease which can be cancer (for example, breast cancer), atherosclerosis or increased vascular risk, rheumatoid arthritis, Alzheimer's disease, amyloidosis, giant cell arthritis, coronary heart disease, Behçet's disease, sickle cell anemia, immune thrombocytopaenic purpura, HIV, stroke, pre-eclampsia, inflammation-associated thrombosis, type II diabetes, or infection. The level of inflammation can also be indicative of state of a disease in the subject. The method (100) can be used to monitor disease progression in the subject by measuring levels of inflammation at different times. This can be particularly useful for monitoring the effect of a therapeutic treatment administered to the subject over time.

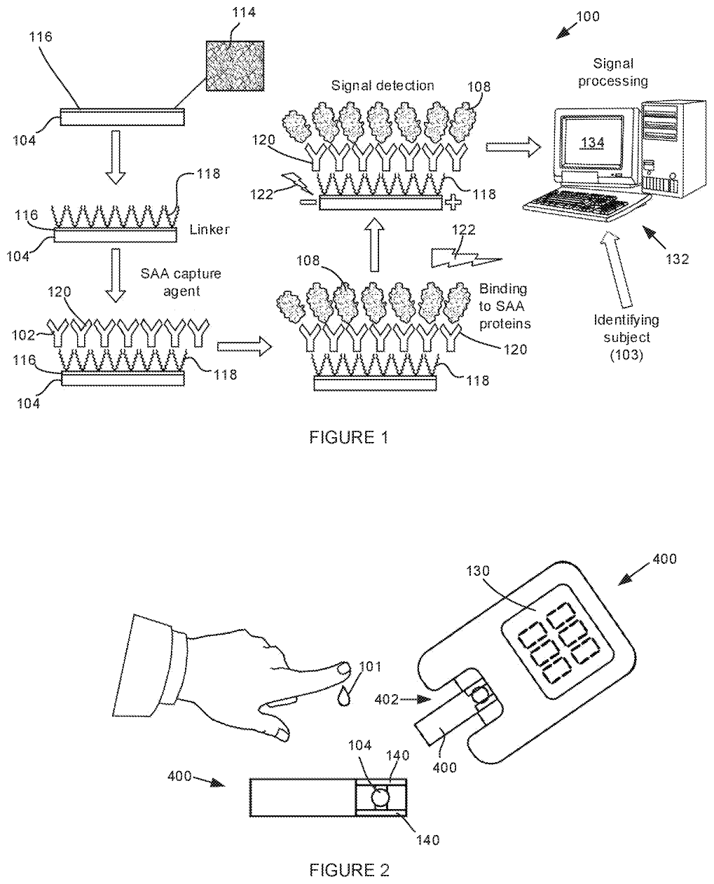

The method (100) is schematically represented in FIG. 1 and includes contacting a biological sample (101) obtained from the subject with a serum amyloid A (SAA) capture agent (102), the capture agent (102) being secured to a substrate, which may be an electrically conductive polymeric nanofibre (110) containing metal nanoparticles, or a piezoelectric nanowire, and which is configured to emit a signal (106), which may be an impedance signal or a piezoelectric signal, upon binding to SAA (108); detecting a signal; and outputting a result indicating a level of inflammation in the subject based on the detected signal (106).

Detection of the signal can include any suitable means of determining whether SAA has bound to the capture agent. This can include measuring the absence of a signal, measuring the presence of a signal, measuring a baseline signal and any deviation from the baseline signal, measuring a positive value signal, measuring a negative value signal, measuring a signal corresponding to an absence of binding of SAA by the capture agent and measuring any signal deviation therefrom, measuring a phase shift signal, measuring a phase difference signal, and measuring a signal corresponding to binding of SAA by the capture agent and measuring any deviation therefrom.

The method can include comparing the detected signal (106) emitted by the capture agent (102) with a predetermined reference value, which can be one or more values on a standard curve, to determine the level of SAA in the sample. The predetermined reference value can be any suitable reference that permits a level of SAA in a sample of unknown concentration to be determined, or which permits a level of inflammation in a subject having an unknown level of inflammation to be determined. Examples include impedance or piezoelectric values obtained for samples of known concentrations of SAA, inflammation indicators obtained from subjects having known levels of inflammation, and impedance or piezoelectric values obtained from samples having known concentrations of other inflammatory markers such as C-reactive protein, fibrinogen, ferritin, α-1-antitrypsin (A1AT), myeloperoxidase (MPO), and soluble tumour necrosis factor-α receptor type II (TNFR II).

The capture agent (102) can be selected from SAA-binding antibodies or antibody fragments, high density lipoprotein (HDL), affibodies, ankyrin repeat proteins, armadillo repeat proteins, nucleic acid aptamers, modified nucleic acid aptamers, peptides, modified peptides, carbohydrate ligands and synthetic ligands.

The method (100) can include identifying (103) the subject in order to assign data obtained by the method (100) to a subject-specific file or folder. The subject can be identified by manual input of a subject identifier (such as the subject's name, date of birth, physical address or subject code) into a device on which the method (100) is carried out, or into a device on which the data is stored or transmitted remotely. Alternatively or in addition, the subject can be identified by a biometric scanner which recognises the subject based on facial, finger print, hand, iris, retina, vein or voice characteristics. Alternatively or in addition, the subject can be identified by a microchip reader, a radiofrequency identification (RFID) reader, a bar code scanner, or a matrix bar code scanner configured to detect a microchip, RFID tag, barcode or matrix barcode corresponding to the subject.

The subject-specific file or folder can be stored on a device on which the method is carried out or it can be stored remotely, such as on a cloud-based server, a remote database, or another computing device. The data can embody a quantity or level of SAA or a quantity or level of inflammation in the subject. Data from a plurality of analyses performed according to the method can be stored in the subject-specific file or folder. The data can be used to analyse levels of SAA or inflammation in the subject over time.

In embodiments in which the substrate is an electrically conductive polymeric nanofibre containing metal nanoparticles, in order to increase the total surface area and durability of the substrate (104), multiple nanofibres can be aggregated to form a textile (114), which can be a woven or a non-woven textile. The nanofibre can be formed by electrospinning a non-electrically conductive first polymer with an electrically conductive second polymer. In a typical embodiment, the first polymer is selected from non-conductive polymers such as polyethylene, polypropylene and polybutylene. The electrically conductive second polymer can be selected from poly(3,4-ethylenedioxythiophene) polystyrene sulfonate (PEDOT:PSS), polyacetylene, polyfluorene, polyphenylene, polyphenylene vinylene, polyphenylene sulfide, polypyrene, polyazulene, polynaphthalene, polypyrrole, polycarbazole, polyindole, polyazepine, polyaniline, polythiophene, and derivatives thereof.

The nanofibre (104) can include metal nanoparticles or a metal coating (116). The metal nanoparticles can be embedded in the nanofibre structure by combination with the first and second polymers during electrospinning. Electrospinning results in an even distribution of the nanoparticles throughout the nanofibre, which ensures a high level of reproducibility of results. Furthermore, electrospinning serves to securely embed the nanoparticles in the nanofibre structure, which limits leaching and loss of the nanoparticles from the nanofibre during use. This may extend the working lifespan of the nanofibre. The metal nano-particles increase the conductivity of the second conducting polymer, thereby decreasing the overall resistance of the nanofibre. This serves to increase the sensitivity of detection and permit smaller impedance signals to be detected. The metal nanoparticles or metal coating can be selected from gold, copper or silver metal, and are preferably gold.

The capture agent (102) may be secured to the metal nanoparticles or metal coating by a linker (118). The linker (118) may include a mercapto functionality at a first end thereof for binding to the metal, and an alkanoic acid at an opposite second end thereof for binding to the capture agent. In some embodiments, the linker is a self-assembled monolayer (SAM). The SAM can be selected from 2-mercaptoethanoic acid, 3-mercaptopropanoic acid, 4-mercaptobutanoic acid, 5-mercaptopenatoic acid, 6-mercaptohexanoic acid, 7-mercaptoheptanoic acid or 8-mercaptooctanoic acid, and is preferably 3-mercaptopropanoic acid. The linker (118) can include a polyethylene glycol spacer between the mercapto and alkanoic acid groups. In some embodiments, the linker (118) is poly(ethylene glycol) 2-mercaptoethyl ether acetic acid and has a number average molecular weight of between 3000 and 4000 g/mol, preferably about 3500 g/mol.

In some preferred embodiments, the capture agent (102) is an SAA-binding antibody or antibody fragment (120), the nanofibre (110) includes gold nanoparticles embedded in its structure, and 3-mercaptopropanoic acid-containing SAMs link the SAA antibodies or antibody fragments to the gold nanoparticles. The signal (106) emitted by the immobilised antibody or antibody fragment (120) upon binding to SAA (108) is in the form of an electrical resistance (impedance) signal (122). When a current, such as a constant current, is operatively applied across the nanofibre, binding of SAA (108) to the antibody or antibody fragment (120) causes electrical impedance to change, by increasing or decreasing. The change in electrical impedance is detectable and can correspond to a level of SAA (108) in the sample (101).

In other embodiments, the capture agent (102) is high density lipoprotein (HDL) or an SAA-binding fragment thereof which is immobilised on the nanofibre (104), as defined above. The signal (106) emitted by the immobilised HDL or fragment (120) upon binding to SAA (108) is in the form of an electrical resistance (impedance) signal (122). For example, the nanofibre substrate (104) can be electrically conductive so that when a current, such as a constant current, is operatively applied thereacross, binding of SAA (108) to the HDL or SAA-binding fragment causes electrical impedance to change, by increasing or decreasing. The change in electrical impedance is detectable and can correspond to a level of SAA (108) in the sample (101).

Typical impedance values in a textile (114) having the nanofibres (110) described above are between 10 and 2500 Ohms (Ω). Impedance signals (122) resulting from binding of SAA (108) to capture agents (102) immobilised on the nanofibre textile (114) typically range from about 1 to about 100Ω, depending on the concentration of SAA (108) in the sample (101).

The method (100) may be capable of detecting picogram, nanogram, or microgram quantities of SAA (108) in the sample (101).

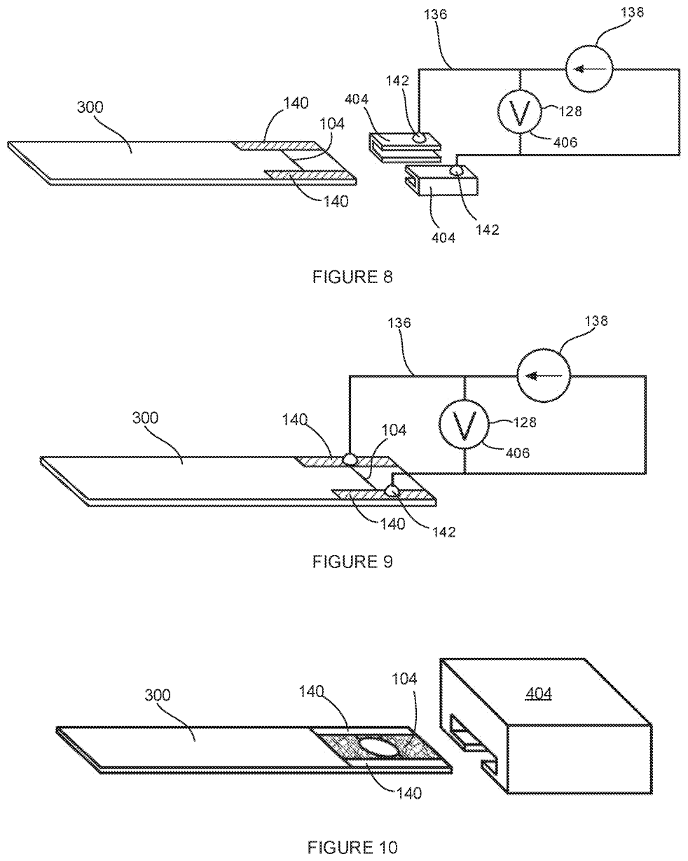

In further embodiments, as illustrated in FIGS. 2, 3 and 6-10, the electrically conductive polymeric nanofibre (110) containing metal nanoparticles can be included in a test strip (300) which may be suitable for use with an inflammation measuring device (400), which can be a point-of-care device, such as a hand held device. In alternative embodiments, the nanofibre (110) can be integrally formed with a sample (101) receiving surface of a point-of-care device, such as a hand held device, in which case the nanofibre (110) is capable of being successively used with multiple samples.

The method (100) can further include amplifying the detected signal (106) to produce an amplified signal; converting the amplified signal to a digital signal; recording, analysing and/or processing the digital signal; determining an amount of SAA in the sample; and assigning a level of inflammation based on the amount of SAA (108) detected.

The biological sample (101) can be whole blood, blood plasma, blood serum, urine, saliva, sputum, or tissue obtained from a biopsy. In a typical example, a blood sample may be allowed to clot before the SAA is detected in the blood serum. Alternatively, an anticoagulant may be added to the blood sample to prevent it from clotting. The blood cells may then be separated and the SAA can be detected in the blood plasma.

The method can permit a therapeutic treatment administered to the subject to be monitored over time. Subject-specific data obtained at different times can be compared to determine the subject's response to the treatment. For example, a level of SAA or inflammation in the subject can be determined before the therapeutic treatment to obtain a pre-treatment level of SAA or inflammation, a level of SAA or inflammation in the subject can be determined after the therapeutic treatment to obtain a post-treatment level of SAA or inflammation, and the pre-treatment and post-treatment levels can be compared to determine the effect of the treatment on inflammation in the subject. The subject's SAA or inflammation levels can be analysed over one or more spaced apart time intervals to determine a trend in the subject's inflammation in response to the treatment. The trend can be graphically represented on a user interface.

The therapeutic treatment can be determined to be successful if the post-treatment level of SAA or inflammation is lower than the pre-treatment level, or if the post-treatment level of SAA or inflammation is higher than the pre-treatment level but lower than would be expected had the treatment had not been performed. This aspect of the invention can be useful for monitoring regression, progression or treatment of a disease involving inflammation associated with upregulated SAA and assessing the effect of therapeutic agents and treatment regimens on the disease. The therapeutic treatment can be any suitable treatment appropriate for the disease. In some embodiments in which the subject suffers from inflammation resulting from cancer, the therapeutic treatment may be radiation therapy, chemotherapy, immunotherapy, targeted therapy, hormone therapy, or stem cell transplant.

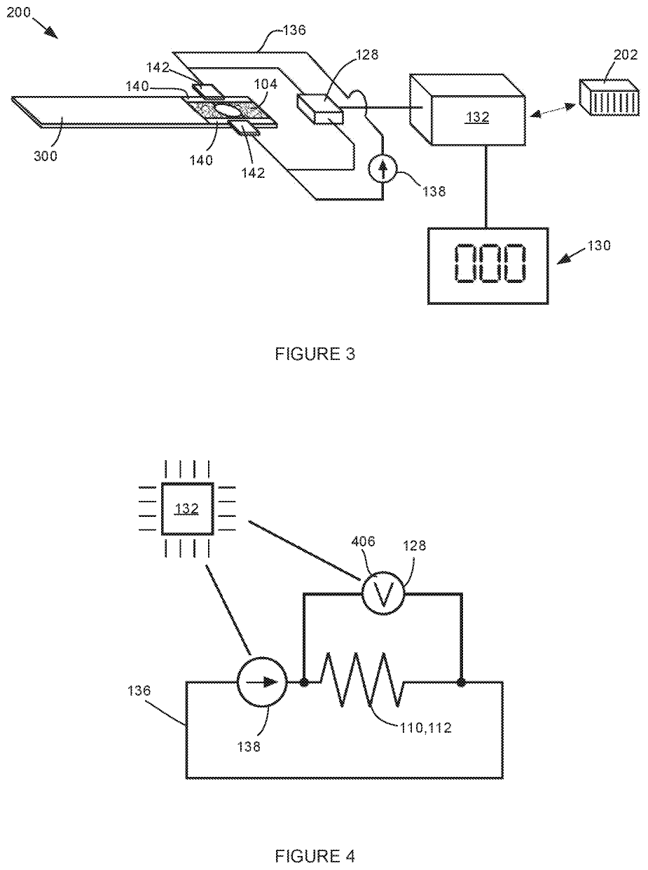

As illustrated in FIGS. 3 and 4, the invention extends to a system (200) for detecting a level of inflammation in a subject according to the method (100) described above. The system (200) can include: a substrate (104) for receiving a biological sample (101) from the subject thereon; a capture agent (102) secured to the substrate (104) for binding SAA (108) in the sample (101), the capture agent (102) configured to emit a signal (106) upon binding to SAA (108); a sensor (128) in communication with the substrate for detecting the emitted signal (106); and an output member (130) in communication with the sensor configured to output a result indicating a level of inflammation in the subject based on the detected signal (106).

The capture agent (102), substrate (104), signal (106) and biological sample (101) can be as defined above.

The system (200) can further include a processor (132) in communication with the sensor (128) for executing several steps of the method, including amplifying the detected signal to produce an amplified signal, converting the amplified signal to a digital signal, recording, analysing and/or processing the digital signal, determining an amount of SAA in the sample, and assigning a level of inflammation based on the amount of SAA detected. The processor (132) can be configured to determine the amount of SAA (108) in the sample by comparing the detected signal (106) with a predetermined reference value, which may be one or more values on a standard curve. A level of inflammation in the subject may then be assigned based on the amount of SAA (108) in the sample (101).

The system (200) may further include software components. The software components can be stored in a memory component (202) and can contain instructions for the processor (132) to execute several of the steps of the method (100). Some or all of the software components may be provided by a software application downloadable onto and executable on a point-of-care device, such as a hand held device.

A storage means, which may be a hard drive or alternatively a remotely accessible storage means, can be provided for storing the detected signal (106), the amount of SAA in the sample, and the assigned level of inflammation.

The output member (130) can include a display means (134), which may be a screen or a graphic user interface, for displaying the amount of SAA detected or the level on inflammation assigned.

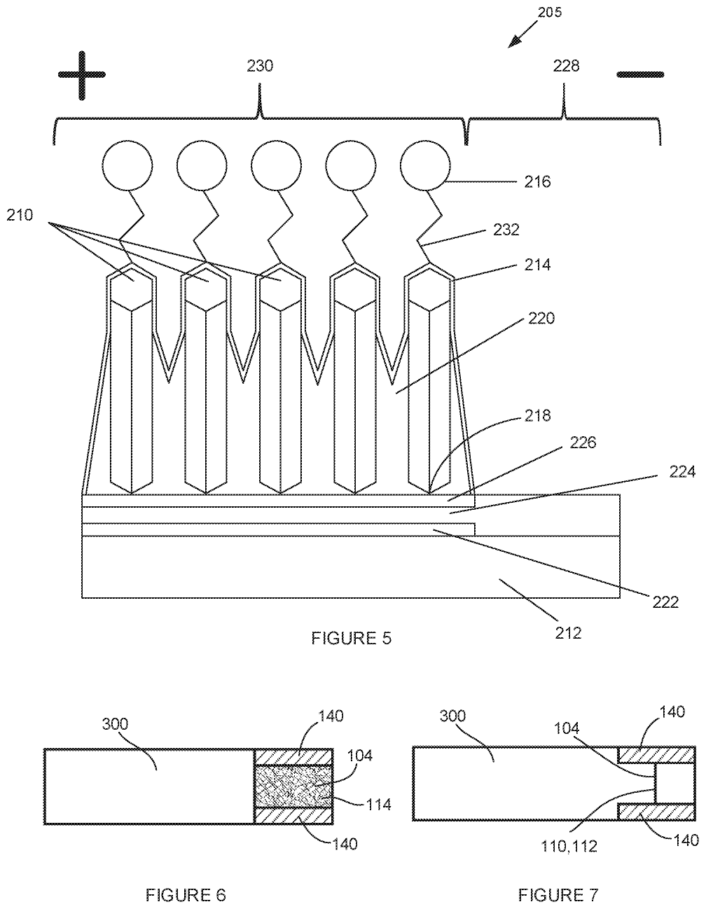

In some embodiments of the system (205), as exemplified in FIG. 5, the substrate can include a plurality of piezoelectric nanowires (210) having ends mounted on a semi conductive substrate (212) and opposite free ends (214) extending generally parallel to each other in a direction substantially perpendicular to the semi conductive substrate (212). Each nanowire (210) can have the capture agent (216) immobilised onto at least a portion of a surface of a free end (214) thereof. In these embodiments, base portions (218) of the nanowires (210) can be coated with an insulating layer (220) of material which may fill the spaces between the nanowires (210) whilst the free ends (214) remain substantially uncoated and uninsulated. Displacement of the nanowires (210) owing to binding of SAA (108) with the capture agent (216) immobilised on the free ends (214) can produce a detectable piezoelectric signal.

The semi conductive substrate (212) can be silicon wafers. A first section of a surface of the silicon wafers can be coated or partially coated with a layer of titanium or titanium oxide (222) which can be approximately 20 nm thick. The titanium/titanium oxide-coated silicon wafers can be further coated with a conductive layer (224), preferably a gold layer that is approximately 40 nm thick. A zinc oxide (ZnO) seed layer (226) can be provided on the gold layer so as to enable the growth of ZnO nanowires onto the substrate. A second section (228) of the surface of the substrate can be coated or partially coated with a conductive layer (224) only, which is preferably a layer of gold. The first section (230) of the surface can act as a cathode (+) in use and the second section (228) of the surface can act as an anode (−) in use.

The ZnO nanowires (210) according to this embodiment can be grown onto the ZnO seed layer so as to extend perpendicularly to the seed layer having a selected length-to-diameter ratio. The base portions (218) of the elongate ZnO nanowires (210) and the ZnO seed layer can be coated with an insulating layer (220) of material, which can be poly(l-vinylpyrrolidone-co-2-dimethylaminoethyl methacrylate), whilst the free ends (214) of the ZnO nanowires (210) remain uncoated and uninsulated. The base portions (218) and free ends (214) of the ZnO nanowires (210) can be coated on at least a portion thereof with a conductive layer (224) of material, which can be a gold coating, preferably a 10 nm gold coating. The capture agent may be secured to the gold coating via a linker (232), which in some embodiments may be provided by glutaraldehyde, and in other embodiments by streptavidin. The streptavidin may be immobilised on the gold coating and may be arranged to bind a biotin molecule on the capture agent.

The system (205) having the ZnO nanowires (210) can be mounted on a board in electronic communication with a measuring system. The measuring system can include a receiver and an amplifier circuit including an operational amplifier that is configured to, in use, amplify a voltage obtained from the piezoelectric signal. The measuring system can be connected to a converter configured to convert the amplified voltage into a digital signal, an operating system with a program that issues machine-readable instructions to record, analyse and process the digital signal, and a user interface for providing access to processed signal data on an electronic device.

The invention further extends to a test strip (300) for use in detecting a level of inflammation in a subject. As illustrated in FIGS. 2, 3 and 6-10, the test strip (300) can include: a substrate (104) for receiving a biological sample (101) from the subject thereon; and a capture agent (102) secured to the substrate (104) for binding SAA (108) in the sample, the capture agent (102) being configured to emit an impedance or piezoelectric signal (106) upon binding to SAA (108). The signal (106) emitted by the capture agent can be indicative of the level of inflammation in the subject. The capture agent (102), substrate (104), signal (106) and biological sample (101) can be as defined above.

Where the capture agent (102) is an SAA antibody or antibody fragment (120), the capture agent (102) can be bound to the substrate through a linker (118), and may be bound to the linker through an amide bond, a triazole ring (formed by click chemistry), or any other suitable immobilisation means. Where the substrate is a nanofibre, ends of the nanofibre (104) can be connectable to a circuit (136) to enable a current, which in some embodiments is a constant current produced by a constant current generator (138), to be passed through the nanofibre (110). The test strip (300) can include electrical contacts (140) at ends of the nanofibre (110) or nanowire (210) and the contacts (140) can be configured to engage corresponding terminals (142) of the circuit (136). In order to increase surface area and durability of the nanofibre (110), some embodiments can include multiple nanofibres aggregated into a woven or non-woven textile (114). Ends of the textile (114) can be secured to the electrical contacts (140). The test strip (300) can be a single-use disposable test strip or a multiple-use test strip and can be suitable for use with an inflammation measuring device (400), which can be a point-of-care device, such as a hand held device.

As shown in FIG. 2, the invention also extends to an inflammation measuring device (400) for detecting a level of inflammation in a subject. The device (400) can include: a sample receiving zone (402) for receiving and contacting a biological sample (101) from the subject with an SAA capture agent (102), the capture agent (102) being secured to a substrate (104) and configured to emit an impedance or piezoelectric signal (106) upon binding to SAA (108); a sensor (128) in communication with the substrate for detecting the emitted signal (106); and an output member (130) in communication with the sensor configured to output a result indicating a level of inflammation in the subject based on the detected signal (106). The capture agent (102), substrate (104), signal (106) and biological sample (101) can be as defined above.

In some embodiments, the sample receiving zone (402) can include a docking means (404) for docking the test strip (300) therein during use. The docking means (404) can be any suitable formation for cooperatively engaging the test strip (300). In embodiments in which the substrate is an electrically conductive polymeric nanofibre containing metal nanoparticles, the device (400) may further include an electrical circuit (136) having a current generator, such as a constant current generator (138), and a resistance detector (406), typically a volt meter or oscilloscope, for detecting resistance in the circuit (136). The circuit (136) can include terminals (142) at ends thereof for cooperatively engaging electrical contacts (140) on the test strip (300). The terminals (142) and/or contacts (140) can include platinum or copper metal.

In other embodiments, the sample receiving zone (402) can include a sample receiving surface integrally formed with the substrate (104) to which the capture agent (102) is secured. In these embodiments, the substrate (104) may be capable of being successively used with multiple samples.

The device (400) may include a processor (132), software components, a memory component (202), output member (130) and/or display means (134) as described above.

The memory component (202) may be configured to store a plurality of subject files corresponding to specific subjects. Each subject file may contain subject-specific data such as the subject's medical records, prior test results, drugs and therapies administered, x-rays, or other reports. In particular, the subject file may contain prior analyses performed using the device. The memory component (202) may be configured to receive data output by the output member in respect of a subject and assign the data to the subject's file. The device (400) may further include an identification means for identifying the subject and correctly assigning the data to the subject's file. The identification means may include a biometric scanner for recognising the subject based on facial, finger print, hand, iris, retina, vein or voice characteristics. Alternatively or in addition, the identification means may include a microchip reader, a radiofrequency identification (RFID) reader, a bar code scanner, or a matrix bar code scanner configured to detect a microchip, RFID tag, barcode or matrix barcode corresponding to the subject. In some embodiments, the test strip can include a subject specific marker capable of being detected by one or more of the aforementioned scanners or readers. The marker on the test strip may include a microchip, RFID tag, bar code or matrix barcode, or another suitable means of identifying the subject.

The display means (134) may be configured to display data contained in the subject's file. The data may include the subject's prior test results, which may be graphically presented to illustrate trends in inflammation in the subject over time.

In some embodiments, a memory component (202) comprising subject-specific files may be located remotely from the device. In these embodiments, the device (400) may be capable of transmitting the inflammation data output by the output member (130) to the remotely located memory component (202).

The device (400) may include an external communications interface for operation of the device (400) in a networked environment enabling transfer of data between multiple computing devices and/or the Internet. Data transferred via the external communications interface may be in the form of signals, which may be electronic, electromagnetic, optical, radio, or other types of signal. The external communications interface may enable communication of data between the device (400) and other computing devices including servers and external storage facilities. Web services may be accessible by and/or from the device (400) via the communications interface.

The external communications interface may be configured for connection to wireless communication channels (e.g. a cellular telephone network, wireless local area network (e.g. using Wi-Fi™), satellite-phone network, Satellite Internet Network, etc.) and may include an associated wireless transfer element, such as an antenna and associated circuitry.

In other embodiments, the device (400) may be connectable to other computing devices by a cable or hardwire.

Computer-readable media in the form of the various memory components (202) may provide storage of computer-executable instructions, data structures, program modules, software units and other data. A computer program product may be provided by a computer-readable medium having stored computer-readable program code executable by a central processor (132). A computer program product may be provided by a non-transient computer-readable medium, or may be provided via a signal or other transient means via the communications interface.

Interconnection via the communication infrastructure (405) allows the one or more processors (132) to communicate with each subsystem or component and to control the execution of instructions from the memory components, as well as the exchange of information between subsystems or components. Peripherals (such as printers, scanners, cameras, or the like) and input/output (I/O) devices (such as a mouse, touchpad, keyboard, microphone, touch-sensitive display, input buttons, speakers and the like) may couple to or be integrally formed with the device (400) either directly or via an I/O controller.

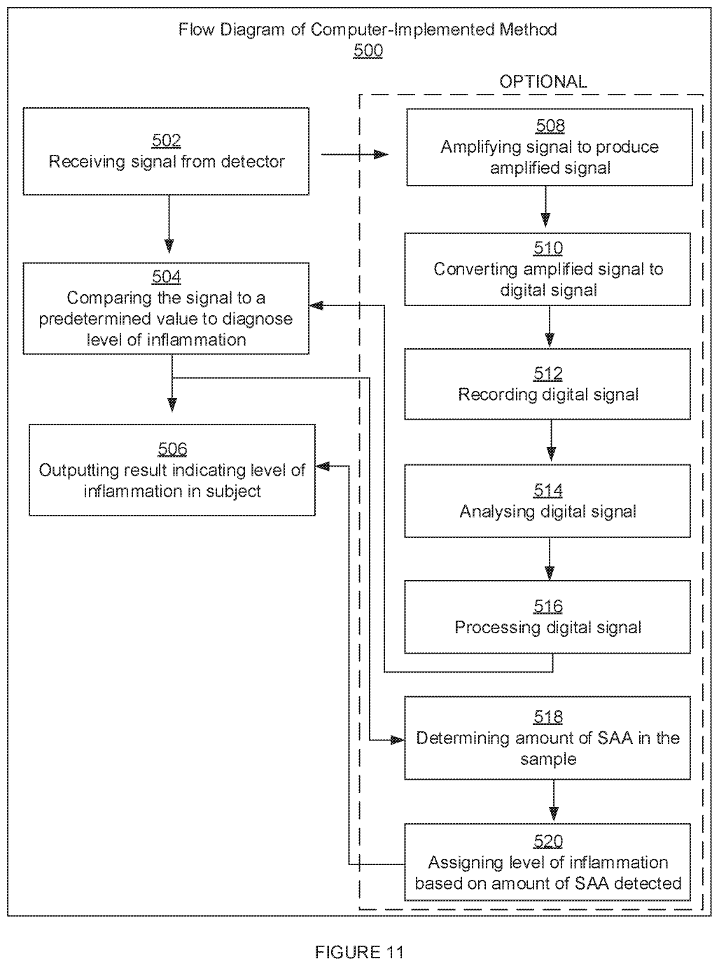

The invention extends even further to a computer-implemented method (500) for detecting inflammation in a subject. As illustrated in FIG. 11, the computer-implemented method (500) can include: receiving (502) a signal from a detector configured to detect binding of SAA (108) in a biological sample (101) to an SAA-binding capture agent (102), in which the capture agent (102) is secured to a substrate (104) and is configured to emit a signal (106) upon binding to SAA (108);

comparing (504) the signal (106) to a predetermined value to diagnose the level of inflammation in the subject; and outputting (506) a result indicating the level of inflammation in the subject based on the signal (106). The capture agent (102), substrate (104), signal (106) and biological sample (101) can be as defined above.

The computer-implemented method (500) can optionally further include amplifying (508) the signal to produce an amplified signal; converting (510) the amplified signal to a digital signal; recording (512), analysing (514) and processing (516) the digital signal; determining (518) an amount of SAA in the sample; and assigning (520) a level of inflammation based on the amount of SAA (108) detected.

The method (100), system (200), test strip (300), device (400), and computer-implemented method (500) according to the invention are significantly more sensitive than existing methods and enable picogram levels of SAA to be detected. Furthermore, diagnosis can be completed in less than a minute. This allows practitioners to diagnose inflammatory responses, early onset of cancer and Alzheimer's disease much faster than existing methods. Furthermore, practitioners are able to follow the progression of the disease during treatment at a fraction of current costs.

The invention will now be described in further detail by way of the following non limiting examples.

### EXAMPLES

### Example 1

A system according to the invention includes an electrically conductive polymeric nanofibre for receiving a biological sample from a subject thereon. The nanofibre contains gold nanoparticles embedded therein and a linker, which may be a 3-mercaptopropanoic acid-containing SAM, securing SAA-binding antibodies or antibody fragments to the gold nanoparticles. In use, a constant current generator applies a constant current to the nanofibre and when SAA in the biological sample binds to the antibodies or antibody fragments resistance in the nanowire increases. The increase in resistance is proportional to the amount of SAA in the sample and can be detected by a detector, typically a volt meter or oscilloscope, as an impedance signal. The system further includes a processor in communication with the detector and configured to carry out the steps of: amplifying the resistance signal, converting the amplified signal to a digital signal, recording the digital signal, analysing the digital signal by comparing it to a standard curve to determine a level of SAA in the sample, and assigning a level of inflammation in the subject based on the level of SAA in the sample. A display screen is further provided for displaying either or both of the amount of SAA detected and the assigned level of inflammation.

### Example 2

A test strip according to the invention includes an electrically conductive polymeric nanofibre for receiving a biological sample from a subject thereon. The nanofibre contains gold nanoparticles embedded therein and a linker, which may be a 3-mercaptopropanoic acid-containing SAM securing SAA-binding antibodies or antibody fragments to the gold nanoparticles. The test strip is configured to be positioned in a sample receiving zone of an inflammation measuring device in such a way that the nanofibre can be connected to a constant current generator. An increase in resistance in the nanowire resulting from binding of SAA to the antibody or antibody fragment is measurable by a resistance detector and a level of SAA in the sample determinable therefrom. A level of inflammation in the subject can then be assigned based on the level of SAA in the sample. The test strip is preferably manufactured to be a single-use, disposable test strip.

### Example 3

A device for use with the test strips of the second example is provided. The device includes a sample receiving zone for receiving the test strip and an electrical circuit to which the test strip is connectable when positioned in the sample receiving zone. The electrical circuit includes a constant current generator and a resistance detector, which is typically a volt meter or oscilloscope, for detecting resistance in the circuit. When the test strip is positioned in the sample receiving zone, a biological sample from a subject can be deposited on the test strip substrate and electrical resistance resulting from binding between SAA in the sample and the capture agent on the substrate detected. The device optionally includes a processor in communication with one or more of the constant current generator, resistance detector, or diode array detector for executing the steps of: amplifying the resistance signal, converting the amplified signal to a digital signal, recording the digital signal, analysing the digital signal by comparing it to a standard curve to determine a level of SAA in the sample, and assigning a level of inflammation in the subject based on the level of SAA in the sample. Software stored on a memory component of the device contains instructions for executing the steps carried out by the processor. A display screen is further provided for displaying either or both of the amount of SAA detected and the assigned level of inflammation.

The foregoing description has been presented for the purpose of illustration; it is not intended to be exhaustive or to limit the invention to the precise forms disclosed. Persons skilled in the relevant art can appreciate that many modifications and variations are possible in light of the above disclosure.

The language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

Throughout the specification unless the contents require otherwise the word ‘comprise’ or variations such as ‘comprises’ or ‘comprising’ will be understood to imply the inclusion of a stated integer or group of integers but not the exclusion of any other integer or group of integers.

1. A method for detecting a level of inflammation in a subject, the method comprising contacting a biological sample obtained from the subject with a serum amyloid A (SAA) capture agent which is secured to an electrically conductive polymeric nanofibre and which is configured to emit an impedance signal upon binding to SAA, detecting a signal, and outputting a result indicating a level of inflammation in the subject based on the detected signal.

2. The method as claimed in claim 1, further comprising comparing the signal with a predetermined reference value to diagnose the level of inflammation in the subject.

3. The method as claimed in claim 1, wherein the capture agent is selected from the group consisting of thioflavins, NIAD-4 (2-[[5′-(4-hydroxyphenyl)[2,2′-bithiophen]-5-yl]-methylene]-propanedinitrile), luminescent conjugated oligothiophene (LCO) markers, SAA-binding antibodies or antibody fragments, high density lipoprotein (HDL), affibodies, ankyrin repeat proteins, armadillo repeat proteins, nucleic acid aptamers, modified nucleic acid aptamers, peptides, modified peptides, carbohydrate ligands, and synthetic ligands.

4. The method as claimed in claim 3, wherein the capture agent is an SAA-binding antibody or antibody fragment.

5. The method as claimed in claim 1, wherein the nanofibre contains metal nanoparticles.

6. (canceled)

7. The method as claimed in claim 5, wherein the metal nanoparticles are gold nanoparticles.

8. The method as claimed in claim 1, wherein self-assembled monolayers (SAMs) are secured to the nanofibre and the capture agent is bound to the SAMs.

9.-12. (canceled)

13. The method as claimed in claim 1, wherein the biological sample is whole blood, blood plasma, blood serum, urine, saliva, sputum, or tissue obtained from a biopsy.

14. A system for detecting a level of inflammation in a subject, the system comprising an electrically conductive polymeric nanofibre for receiving a biological sample from the subject thereon, a capture agent secured to the nanofibre for binding serum amyloid A (SAA) in the sample, the capture agent being configured to emit an impedance signal upon binding to SAA, a sensor in communication with the nanofibre for detecting the emitted signal, and an output member in communication with the sensor configured to output a result indicating a level of inflammation in the subject based on the detected signal.

15. The system as claimed in claim 14, wherein the nanofibre contains metal nanoparticles.

16. (canceled)

17. The system as claimed in claim 15, wherein the metal nanoparticles are gold nanoparticles.

18. The system as claimed in claim 14, wherein self-assembled monolayers (SAMs) are secured to the nanofibre and the capture agent is bound to the SAMs.

19. (canceled)

20. The system as claimed in claim 14, wherein the sensor is selected from a volt meter, an ammeter, an oscilloscope and a power meter.

21. The system as claimed in claim 14, wherein the nanofibre is included in a test strip configured for use with a point-of-care device.

22. A test strip for use in detecting a level of inflammation in a subject, the test strip including an electrically conductive polymeric nanofibre for receiving a biological sample from the subject thereon, and a capture agent secured to the nanofibre for binding serum amyloid A (SAAB in the sample, the capture agent being configured to emit an impedance signal upon binding to SAA when connected to an electrical circuit, the signal being indicative of the level of inflammation in the subject.

23. The test strip as claimed in claim 22, which is configured for use with a point-of-care device.

24.-39. (canceled)

40. The method as claimed in claim 1, wherein the nanofibres are piezoelectric nanowires having ends thereof mounted on a semi-conductive substrate and opposite free ends extending generally parallel in a direction substantially perpendicular to the semi-conductive substrate, with each nanowire having the capture agent immobilized onto at least a portion of a surface of a free end thereof, the nanowires being configured to produce a piezoelectric signal when displaced during binding of SAA to the capture agent.

41. The system as claimed in claim 14, wherein the nanofibres are piezoelectric nanowires having ends thereof mounted on a semi-conductive substrate and opposite free ends extending generally parallel in a direction substantially perpendicular to the semi-conductive substrate, with each nanowire having the capture agent immobilized onto at least a portion of a surface of a free end thereof, the nanowires being configured to produce a piezoelectric signal when displaced during binding of SAA to the capture agent.

42. The test strip as claimed in claim 22, wherein the nanofibres are piezoelectric nanowires having ends thereof mounted on a semi-conductive substrate and opposite free ends extending generally parallel in a direction substantially perpendicular to the semi-conductive substrate, with each nanowire having the capture agent immobilized onto at least a portion of a surface of a free end thereof, the nanowires being configured to produce a piezoelectric signal when displaced during binding of SAA to the capture agent.

43. The test strip as claimed in claim 42, base portions of the nanowires are coated with an insulating layer of material which fills the spaces between the nanowires whilst the free ends remain substantially uncoated and uninsulated, the nanowires being configured to produce a piezoelectric signal when displaced during binding of SAA to the capture agent, and at least a portion of the free ends are coated in gold and the capture agent is secured to the gold via a glutaraldehyde linker or a streptavidin linker.
